(19) United States
(12) Patent Application Publication
(10) Pub. No.: US 2023/0255824 A1
(43) Pub. Date: Aug. 17, 2023

(54) TIP UNCLOGGING USING CONTROLLED ASPIRATION REFLUX

(71) Applicant: JOHNSON & JOHNSON SURGICAL VISION, INC., Irvine, CA (US)

(72) Inventors: Vadim Gliner, Haifa (IL); Alon Boumendil, Givat Nili (IL)

(21) Appl. No.: 17/670,734

(22) Filed: Feb. 14, 2022

(51) Int. Cl. A61F 9/007; A61M 1/00

(52) U.S. Cl. CPC A61F 9/00781; A61M 1/73; A61M 1/774; A61M 2205/3331; A61M 2210/0612

(57) ABSTRACT

A phacoemulsification system includes a phacoemulsification probe with a distal end for insertion into an eye of a patient, an irrigation pump, an aspiration pump, and a processor. The probe includes an irrigation channel, an aspiration channel, an irrigation sensor, and an aspiration sensor. The irrigation pump is configured to flow irrigation fluid to the irrigation channel. The aspiration pump is configured to evacuate material from the aspiration channel. The processor is configured to detect a clogging of the aspiration channel using the aspiration sensor, to estimate an intra-ocular pressure (IOP) of the eye using the irrigation sensor, to set a reflux pressure for the aspiration pump depending on the estimated IOP, and to repel the clogging by controlling the aspiration pump to apply the reflux pressure.

### FIELD OF THE DISCLOSURE

The present disclosure relates generally to phacoemulsification apparatuses and probes, and particularly to systems for aspiration control.

### BACKGROUND OF THE DISCLOSURE

A cataract is a clouding and hardening of the eye’s natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient’s vision.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification apparatus, in accordance with an example of the present disclosure;

FIG. 2 is a block diagram schematically describing a system of the phacoemulsification apparatus of FIG. 1, the system configured to release a clog in a controlled manner by altering aspiration flow, in accordance with an example of the present disclosure; and

FIG. 3 is a flow chart schematically illustrating a method for repelling a clog in a hollow needle of the phacoemulsification apparatus of FIG. 1, in accordance with an example of the present disclosure.

### DETAILED DESCRIPTION OF EXAMPLES

### Overview

During phacoemulsification, emulsified lens particles are aspirated through an aspiration tip (e.g., a hollow needle) of a phacoemulsification probe, then via an aspiration channel of the probe and further proximally into an aspiration line. When a particle clogs (e.g., blocks) the inlet of the aspiration tip, the vacuum increases in the aspiration channel and the aspiration line. When the inlet later becomes unblocked (e.g., when the particle is subsequently sucked into the aspiration line), the high vacuum in the line causes an aspiration surge with potentially traumatic consequences to the eye.

It may be possible to release the clog by venting the aspiration channel (e.g., by reducing the vacuum) or even by inducing reverse flow in the channel to expel the clog from the inlet. The problem with these approaches, however, is that if the vent flow is too low, the blockage will not be released, and if the flow is too high, the particle will be repelled with a very high speed which may cause damage to the eye. The actual reverse flow rate needed depends on a plurality of parameters that may not be known. For example, the flow rate needed to release viscous matter is different than the flow rate required to release a brittle particle.

Examples of the present disclosure that are described hereinafter release the clog in a controlled manner. In one example, once an occlusion is detected (indicative of clogging) (e.g., an increase in vacuum in the aspiration line/channel), a processor of the phacoemulsification apparatus alters (e.g., reverses) the aspiration flow in the aspiration line/channel to generate a slight overpressure inside the hollow needle above the outside intraocular pressure (IOP), with a given ΔP (e.g., 20 mmHg). The given ΔP is selected to be sufficient to repel the clogging particle but not to cause damage to the eye. To this end, the processor monitors the IOP in real time. For example, the processor estimates the IOP using the irrigation pressure sensor and an empirical offset, since the irrigation pressure is measured at the proximal end of the handpiece.

After a predefined time duration of the slight overpressure, which ensures that the particle is removed, the processor returns the aspiration pump to its nominal aspiration mode to reestablish a nominal aspiration vacuum (i.e., sub-pressure), such as to a previously pre-programmed aspiration vacuum level.

In an example, a phacoemulsification system, is provided, which includes a phacoemulsification probe configured for insertion into an eye of a patient, the probe comprising (a) an irrigation channel for irrigating the eye with irrigation fluid, and (b) an aspiration channel for evacuating material from the eye. The system further includes (a) an irrigation sensor, which is coupled with the irrigation channel and is configured to measure a parameter indicative of a pressure of the irrigation fluid; and (b) an aspiration sensor, which is coupled with the aspiration channel and is configured to measure a value indicative of a pressure in the aspiration channel. An irrigation pump of the system is configured to flow the irrigation fluid to the irrigation channel. An aspiration pump of the system is configured to evacuate the material from the aspiration channel. A processor of the system is configured to detect a clogging of the aspiration channel using the aspiration sensor, to estimate an IOP of the eye using the irrigation sensor, to set a reflux pressure for the aspiration pump depending on the estimated IOP, and to repel the clogging by controlling the aspiration pump to apply the set reflux pressure. The processor is configured to set the reflux pressure to be a predefined pressure above the estimated IOP.

In particular, the processor is configured to repel the clogging by reversing a flow direction in the aspiration pump and then applying the set reflux pressure for the aforementioned predefined time duration.

### Apparatus Description

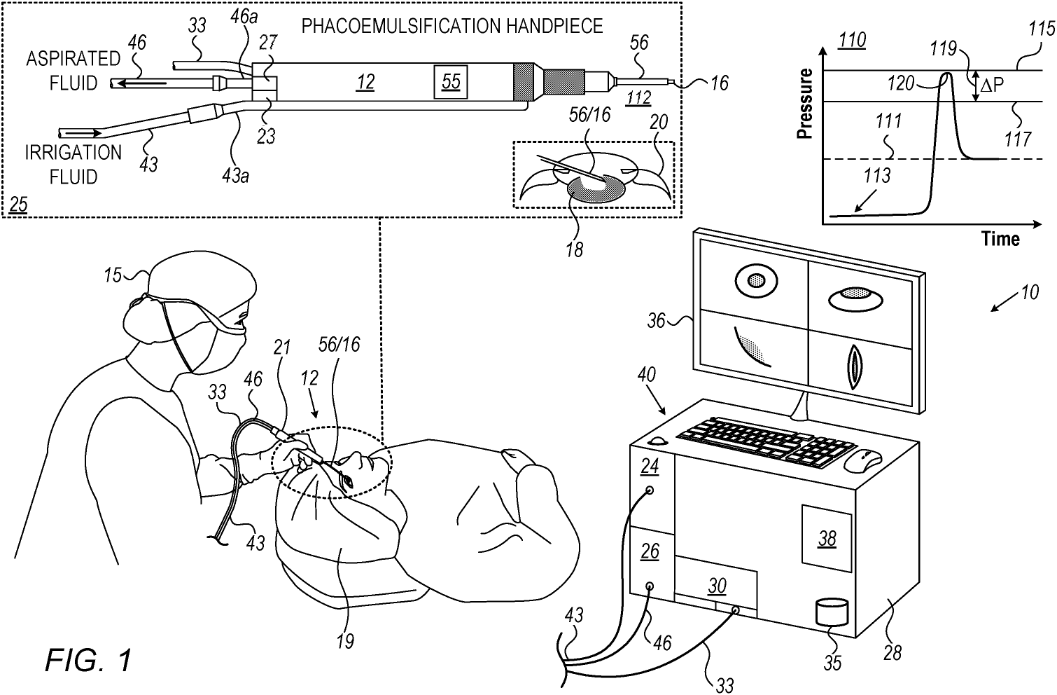

FIG. 1 is a schematic, pictorial view, along with an orthographic side view, of a phacoemulsification apparatus 10, in accordance with an example of the present disclosure.

As seen in the pictorial view of phacoemulsification apparatus 10, and in the orthographic side view inset 25, a phacoemulsification probe 12 (e.g., a handpiece) comprises a distal end 112 comprising a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure a processor-controlled irrigation pump 24 comprised in a console 28 pumps irrigation fluid from an irrigation reservoir (not shown) to the irrigation sleeve 56 to irrigate the eye. The fluid is pumped via an irrigation tubing line 43 running from console 28 to an irrigation channel 43a of probe 12. In another example, pump 24 may be coupled with or replaced by a gravity-fed irrigation source such as a balanced salt solution bottle/bag.

Eye fluid and waste matter (e.g., emulsified parts of the cataract) are aspirated via hollow needle 16 to a collection receptacle (not shown) by a processor-controlled aspiration pump 26, also comprised in console 28, using aspiration tubing line 46 running from aspiration channel 46a of probe 12 to console 28. In an example, processor 38 controls an aspiration rate of aspiration pump 26 to maintain intraocular pressure (in case of sub-pressure indicated, for example, by sensor 27) within prespecified limits.

Channels 43a and 46a are coupled respectively with irrigation line 43 and aspiration line 46. Pumps 24 and 26 may be any pump known in the art (e.g., a peristaltic pump). Using sensors (e.g., as indicated by sensors 23 and/or 27), processor 38 controls a pump rate of irrigation pump 24 and aspiration pump 26 to maintain intraocular pressure (IOP) within prespecified limits.

In the shown example, probe 12 includes an irrigation sensor 23 coupled with irrigation channel 43a and an aspiration sensor 27 coupled with an aspiration channel 46a. In order to release a clog in needle 16 in a controlled manner, processor 38 monitors the intraocular pressure (IOP) as it alters the aspiration flow in needle 16. The clog is removed by reversing (refluxing) the aspiration flow to achieve a pressure inside channel 46a that is up to a given ΔP (e.g., 20 mmHg) above the IOP. Processor 38 estimates the IOP using readings from the irrigation pressure sensor 23 and an empirical offset, since the irrigation pressure is measured at the proximal end of handpiece 12.

An example of the unclogging of needle 16 is given in graph 110, a pressure reading from sensor 27. The disclosed technique aims at restoring a vacuum level from an occlusion vacuum level 113 (increased vacuum level) (caused by the clog) to a nominal vacuum level 111. By refluxing, aspiration pump 26 generates a moderate pressure pulse with a peak pressure 115 over a duration 120, which is ΔP 119 (e.g., 20 mmHg) above a measured IOP 117. A finite duration 120 of up to few seconds is required, since the pressure buildup is minor (e.g., 20 mmHg) which takes time to build at the tip of needle 16, which is typically located 15 cm distally to where the reading by sensor 27 is taken. The pressure releases the clog, and the subsequent operation of the aspiration pump in its nominal aspiration mode brings aspiration to the nominal sub-pressure value 111 (e.g., to 350 mmHg). In another example, not shown, instead of applying the pressure for a predefined time duration 120, the processor may detect that the clog was repelled by receiving, in real time, a change in reading of sensor 27 (e.g., a small drop from peak 115 to IOP level 117 when the clog is repelled) and only then revert the operation of aspiration pump 26 to obtain nominal sub-pressure (e.g., aspiration vacuum) value 111.

In one example, the control of aspiration pump 26 is done with a proportional-integral-derivative (PID) controller. Such a control sets pressure targets (as read by sensor 27), and the pump 26 operates with its own control circuitry (e.g., of rate and direction) to achieve the pressure targets. For example, the PID controller sets a positive target pressure of (IOP+ΔP), whereas ΔP<20 mmHg to regulate the flow rate is required to safely release the clog.

Sensors 23 and 27 may be any sensor known in the art, including, but not limited to, a vacuum sensor or flow sensor. The sensor measurements (e.g., pressure, vacuum, and/or flow) are taken close to the proximal end of the handpiece where the irrigation outlet and the aspiration inlet are located, so as to provide processor 38 with an accurate indication of the actual measurements occurring within an eye and provide a short response time to a control loop comprised in processor 38.

A vacuum sensor, as discussed herein, includes types of pressure sensors that are configured to provide sufficiently accurate measurements of low sub-atmospheric pressures within a typical sub-pressure range at which aspiration is applied (e.g., between 1 mmHg and 650 mmHg). In an example, the same pressure sensor model is used to measure irrigation pressure and aspiration sub-pressure, using different sensor settings/calibrations.

As further shown, phacoemulsification probe 12 includes a piezoelectric crystal 55, coupled to a horn (not shown), that drives needle 16 to vibrate in a resonant vibration mode that is used to break a cataract into small pieces during a phacoemulsification procedure. Console 28 comprises a piezoelectric drive module 30, coupled with the piezoelectric crystal, using electrical wiring running in cable 33. Drive module 30 is controlled by a processor 38 that uses the drive signal or a small-amplitude monitoring signal (e.g., at a detuned frequency) via cable 33 and enables an electrical impedance of crystal 55 to be monitored, to detect an occlusion and perform preemptive steps, such as adjusting the irrigation rate and/or adjusting the aspiration rate to prevent a subsequent vacuum surge.

Processor 38 further conveys processor-controlled driving signals via cable 33 to, for example, maintain needle 16 at maximal vibration amplitude. The drive module may be realized in hardware or software, for example, in a proportional-integral-derivative (PID) control architecture.

Processor 38 may receive user-based commands via a user interface 40, which may include setting a vibration mode and/or frequency of the piezoelectric crystal, and setting or adjusting an irrigation and/or aspiration rate of the irrigation pump 24 and aspiration pump 26. Processor 38 may receive user-based commands via a user interface 40, which may include needle 16 stroke amplitude settings and turning on irrigation and/or aspiration. In an example, the physician uses a foot pedal (not shown) as a means of control. For example, foot pedal position one activates only irrigation, pedal position two activates both irrigation and aspiration, and pedal position three adds needle 16 vibration. Additionally, or alternatively, processor 38 may receive the user-based commands from controls located in a handle 21 of probe 12.

In an example, user interface 40 and display 36 may be integrated into a touch screen graphical user interface.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements, which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

### Repelling a Clog in an Aspiration Tip Using Aspiration Reflux

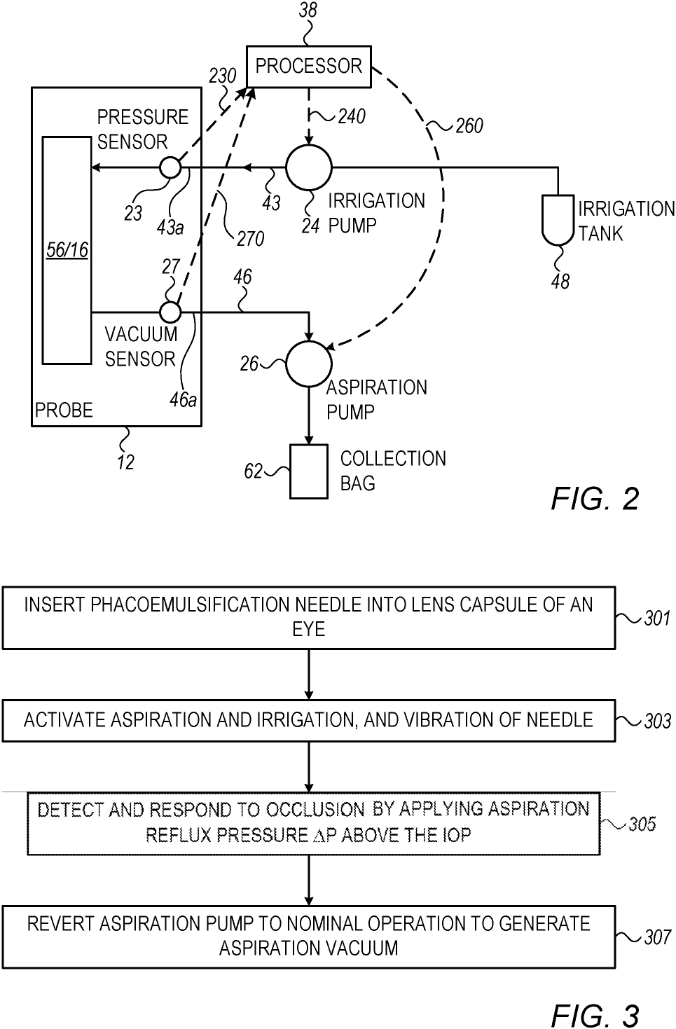

FIG. 2 is a block diagram schematically describing a system of the phacoemulsification apparatus 10 of FIG. 1, which is configured to release a clog in a controlled manner by altering aspiration flow, in accordance with an example of the present disclosure. As seen, processor 38 controls (260) aspiration pump 26 to aspirate eye fluid into a collection bag 62, via aspiration line 46.

As further seen, sensor 23 and sensor 27 of probe 12 provide readings 230 (e.g., pressure, vacuum, or flow) of irrigation channel 43a, and readings 270 (e.g., pressure, vacuum, or flow) of aspiration channel 46a, respectively, to processor 38. The readings (230, 270) are provided in real time at a sufficiently high rate (e.g., 1 kHz) to allow a fast system response time (e.g., within several milliseconds). In the shown example, sensors 23 and 27 are seen as located in the back of handpiece 12. In general, the sensors can be located in a case or module coupled with the handpiece (e.g., by including the sensors in a disposable case coupled to the aspiration and irrigation lines just proximally of the handle itself) or located anywhere along the handpiece 12.

In response to readings (230, 270), processor 38 can change a direction and adjust a rate of aspiration pump 26 to maintain readings (230, 270) within prespecified limits. This capability is manifested, for example, in graph 110 (FIG. 1) of aspiration channel 46a pressure. As seen, upon detecting an occlusion (e.g., vacuum level increase) 113 (using sensor 27), processor 38 commands aspiration pump 26 to work in a reflux mode (including a reversed pumping direction), to generate a pressure pulse with a peak pressure 115, which is ΔP 119 (e.g., 20 mmHg) above a measured IOP 117. The small over-pressure (IOP+ΔP) releases the clog.

After a predefined time duration (e.g., time duration 120), processor 38 commands aspiration pump 26 to return to its nominal aspiration mode, so as to bring aspiration vacuum to the nominal sub-pressure value 111 (e.g., to 350 mmHg).

The example block diagram shown in FIG. 2 is highly simplified and chosen purely for the sake of conceptual clarity. A typical system may include, for example, a complex aspiration apparatus comprising two or more pumps, including a venturi pump. A phacoemulsification system, such as shown in of FIG. 1, additionally includes bypass protection, and may further include valves in irrigation/or aspiration lines.

These and other details of an actual system are omitted for simplicity of presentation. In this regard, irrigation pump 24 represents an irrigation subsystem and aspiration pump 26 represents an aspiration subsystem, with their disclosed functionality described in essence above.

The term “sensor” includes any type of sensor that can provide indications to the processor running the control loop. For the aspiration channel, such a sensor may be a pressure sensor that is configured to provide sufficiently accurate measurements of low sub-atmospheric pressures that are within a typical range of sub-pressures at which aspiration is applied (e.g., between 1 mmHg and 650 mmHg). In an example, sensors 23 and 27 comprise the same pressure sensor model, with different settings/calibrations to measure either irrigation pressure or aspiration sub-pressure. For the irrigation channel, such a sensor may be the aforementioned pressure sensor, or a fluid flow rate meter.

In the example brought by FIG. 2, processor 38 optionally controls (240) irrigation pump 24 to pump a balanced salt solution from an irrigation tank 48 to sleeve 56 (shown in FIG. 1), via irrigation line 43. However, the disclosed technique does not depend on irrigation control. Furthermore, pumps 24 and 26 may include means to indicate actual pump performance (e.g., speed) to processor 38 (not shown in FIG. 2), for use by a feedback loop, which again can be an additional, though not a necessary, feature of the system described by the block diagram of FIG. 2.

FIG. 3 is a flow chart schematically illustrating a method for repelling a clog in a hollow needle 16 of the phacoemulsification apparatus 10 of FIG. 1, in accordance with an example of the present disclosure. The process begins with physician 15 inserting phacoemulsification needle 16 of probe 12 into a lens capsule 18 of an eye 20, at a phacoemulsification needle insertion step 301.

At a phacoemulsification step 303, physician 15 presses a foot pedal to a first position to activate irrigation and subsequently to a second position to activate aspiration, and finally, when the foot pedal is pressed and placed in a third position, the needle 16 is vibrated to perform the phacoemulsification.

During phacoemulsification, processor 38 receives readings from sensor 27 so that, once an occlusion is detected (as indicated by increase in vacuum), processor 38 can react in real time to operate aspiration pump 26 in a reflux mode to generate a slight over-pressure inside the clogged needle 16, i.e., just above IOP (e.g., by ΔP=20 mmHg), so as to repel the clogging particle, at a clog repulsion step 305. The buildup of the gentle over-pressure lasts a predefined time duration 120.

Finally, after predefined time duration 120, processor 38 commands aspiration pump to operate so as to bring the vacuum in aspiration channel 46a (as read by sensor 27) to nominal value 111 (e.g., 350 mmHg), at aspiration restoration step 307.

The steps of FIG. 3 are brought as an example. As another example, instead of using a predefined time duration, processor 38 may command step 307 based on a reading from sensor 27 that is indicative that unclogging occurred, as described above.

### Example 1

A phacoemulsification system (10), comprising a phacoemulsification probe (12), an irrigation pump (24), an aspiration pump (26), and a processor (38). The phacoemulsification probe (12) has a distal end (112) configured for insertion into an eye (20) of a patient (19), the probe comprising an irrigation channel (43a) for irrigating the eye with irrigation fluid, and an aspiration channel (46a) for evacuating material from the eye. An irrigation sensor (23) is coupled with the irrigation channel (43a) and is configured to measure a parameter indicative of a pressure of the irrigation fluid. An aspiration sensor (27) is coupled with the aspiration channel (46a) and is configured to measure a value indicative of a pressure in the aspiration channel. An irrigation pump (24) is configured to flow the irrigation fluid to the irrigation channel (43a). An aspiration pump (26) is configured to evacuate the material from the aspiration channel. The processor (38) is configured to detect a clogging of the aspiration channel (46a) using the aspiration sensor (27), to estimate an intra-ocular pressure (IOP) of the eye using the irrigation sensor (23), to set a reflux pressure for the aspiration pump (26) based on the estimated IOP, and to repel the clogging by controlling the aspiration pump (26) to apply the reflux pressure.

### Example 2

The apparatus according to example 1, wherein the processor is configured to set the reflux pressure to be a predefined pressure above the estimated IOP.

### Example 3

The apparatus according to example 1, wherein the processor is configured to reverse a flow direction in the aspiration pump and then apply the set reflux pressure for a predefined time duration to repel the clogging.

### Example 4

The apparatus according to any one of examples 1 through 3, wherein the processor is further configured to detect that the clogging was repelled based on a reading from the aspiration sensor, and to restore nominal operation of the aspiration pump.

### Example 5

The apparatus according to any one of examples 1 through 4, wherein the probe distal end comprises a hollow needle, and wherein the aspiration channel traverses an internal lumen of the needle.

### Example 6

A phacoemulsification method includes inserting into an eye of a patient a distal end of phacoemulsification probe, the probe comprising: an irrigation channel for irrigating the eye with irrigation fluid; an aspiration channel for evacuating material from the eye; an irrigation sensor, which is coupled with the irrigation channel and is configured to measure a parameter indicative of a pressure of the irrigation fluid; and an aspiration sensor, which is coupled with the aspiration channel and is configured to measure a value indicative of a pressure in the aspiration channel. Using an irrigation pump, the irrigation fluid is flowing to the irrigation channel. Using an aspiration pump, the material is evacuated from the aspiration channel. A clogging of the aspiration channel is detected using the aspiration sensor. An intra-ocular pressure (IOP) of the eye is estimated using the irrigation sensor. A reflux pressure is set for the aspiration pump based on the estimated IOP, and the clogging is repelled by controlling the aspiration pump to apply the reflux pressure.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A phacoemulsification system, comprising:
a phacoemulsification probe having a distal end configured for insertion into an eye of a patient, the probe comprising:
an irrigation channel for irrigating the eye with irrigation fluid;
an aspiration channel for evacuating material from the eye;
an irrigation sensor, which is coupled with the irrigation channel and is configured to measure a parameter indicative of a pressure of the irrigation fluid; and
an aspiration sensor, which is coupled with the aspiration channel and is configured to measure a value indicative of a pressure in the aspiration channel;
an irrigation pump configured to flow the irrigation fluid to the irrigation channel;
an aspiration pump configured to evacuate the material from the aspiration channel; and
a processor, which is configured to detect a clogging of the aspiration channel using the aspiration sensor, to estimate an intra-ocular pressure (IOP) of the eye using the irrigation sensor, to set a reflux pressure for the aspiration pump based on the estimated IOP, and to repel the clogging by controlling the aspiration pump to apply the reflux pressure.

2. The system according to claim 1, wherein the processor is configured to set the reflux pressure to be a predefined pressure above the estimated IOP.

3. The system according to claim 1, wherein the processor is configured to reverse a flow direction in the aspiration pump and then apply the set reflux pressure for a predefined time duration to repel the clogging.

4. The system according to claim 1, wherein the processor is further configured to detect that the clogging was repelled based on a reading from the aspiration sensor, and to restore nominal operation of the aspiration pump.

5. The system according to claim 1, wherein the distal end comprises a hollow needle, and wherein the aspiration channel traverses an internal lumen of the needle.

6. A phacoemulsification method, comprising:
inserting into an eye of a patient a distal end of phacoemulsification probe, the probe comprising:
an irrigation channel for irrigating the eye with irrigation fluid;
an aspiration channel for evacuating material from the eye;
an irrigation sensor, which is coupled with the irrigation channel and is configured to measure a parameter indicative of a pressure of the irrigation fluid; and
an aspiration sensor, which is coupled with the aspiration channel and is configured to measure a value indicative of a pressure in the aspiration channel;
using an irrigation pump, flowing the irrigation fluid to the irrigation channel;
using an aspiration pump, evacuating the material from the aspiration channel;
detecting a clogging of the aspiration channel using the aspiration sensor;
estimating an intra-ocular pressure (IOP) of the eye using the irrigation sensor; and
setting a reflux pressure for the aspiration pump based on the estimated IOP, and repelling the clogging by controlling the aspiration pump to apply the reflux pressure.

7. The method according to claim 6, wherein setting the reflux pressure comprises setting the reflux pressure to be a predefined pressure above the estimated IOP.

8. The method according to claim 6, wherein setting the reflux pressure comprises reversing a flow direction in the aspiration pump and then applying the reflux pressure for a predefined time duration to repel the clogging.

9. The method according to claim 6, further comprising detecting that the clogging was repelled based on a reading from the aspiration sensor, and restoring nominal operation of the aspiration pump.

10. The method according to claim 6, wherein the distal end comprises a hollow needle, and wherein the aspiration channel traverses an internal lumen of the needle.
